(19) United States
(12) Patent Application Publication
(10) Pub. No.: US 2010/0279324 A1
(43) Pub. Date: Nov. 4, 2010

(54) Assay For Detecting Mycobacterial Infection

(75) Inventors: Ajit Lalvani, London (GB); Damien J.C. Montamat-Sicotte, Birmingham (GB); Benjamin E. Willcox, Birmingham (GB); Gurdyal S. Besra, Birmingham (GB)

Correspondence Address: RATNERPRESTIA, P.O. BOX 980, VALLEY FORGE, PA, 19482, US

(73) Assignee: The University of Birmingham, Birmingham (GB)

(21) Appl. No.: 12/599,406

(22) PCT Filed: May 8, 2008
(86) PCT No.: PCT/GB08/01596

§ 371 Date: Jul. 1, 2010

(30) Foreign Application Priority Data
May 8, 2007 (GB) 0708874.3

(51) Int. Cl. G01N 33/53

(52) U.S. Cl. 435/7.24

(57) ABSTRACT

Methods for assessing a mycobacterial infection in a subject comprise exposing at least one CD1 molecule or analogue to mycolic acid or a mycolic acid analogue, subsequently incubating the at least one CD1 molecule or analogue with a sample comprising at least one T cell isolated from the subject, and measuring the T cell response and/or the number of mycolic acid specific T cells present in the T cell sample.

### SUMMARY

The current invention relates to an assay for a detecting mycobacterial infection in a subject by detecting ex vivo T cells specific for mycolic acid antigens.

### BACKGROUND

Mycobacterial infection in the form of M. tuberculosis is a major problem worldwide, with one new infection every second. Tuberculosis is a chronic, infectious disease, that is generally caused by infection with Mycobacterium tuberculosis.

It is a major disease in developing countries, as well as an increasing problem in developed areas of the world. Overall, one-third of the world's population is currently infected with tuberculosis bacilli. 5-10% of otherwise healthy people who are infected with M. tuberculosis become sick or infectious at some time during their life. However, immunocompromised people, such as those with HIV, who are also infected with M. tuberculosis are much more likely to develop TB.

Although the infection may be asymptomatic for a considerable period of time, it may reactivate resulting in a disease that is most commonly manifested as a chronic inflammation of the lungs, resulting in fever and a cough. If left untreated, serious complications and death typically result. Current estimates suggest that there are 9 million new cases of TB per year and almost 2 million deaths. For every patient definitively diagnosed with TB, many more are evaluated for suspected TB (World Health Organization. 2006).

A definitive diagnosis of active TB is made by culturing the TB bacterium, M. tuberculosis, from clinical specimens. However, culture results take 2-8 weeks to become positive and, in a substantial minority of patients (20-50%) cultures are negative. Therefore, alternative, newer diagnostic tests for active TB are urgently required (reviewed in Dinnes et al, Health Technol Assess. 2007).

Some tests of TB infection test for sensitisation of the cellular immune system to M. tuberculosis proteins. The most widely-used of these is the tuberculin skin test (also known as the PPD test or Mantoux test or Tuberculin Sensitivity Test or Pirquet test) which is over 100 years old. This test is based on identifying a response to exposure to tuberculin which is a glycerine extract of the tubercule bacilli. The standard material used in this test is purified protein derivative tuberculin (PPD) which is a precipitate of non-species-specific molecules obtained from filtrates of sterilized, concentrated cultures.

The test comprises intradermal injection of a standard dose of 5 Tuberculin units (0.1 ml) into the volar aspect of the forearm. The results are obtained through clinical examination 48 to 72 hours later. A person who has been exposed to the bacteria is expected to mount a delayed type hypersensitivity immune response in the skin containing the bacterial proteins. Whereas, no response will be seen in individuals who have not been exposed to TB (http://www.cdc.gov/nchstp/tb/pubs/Mantoux/part1.htm).

This test has a number of problems, firstly, it requires re-examination of the patient 3 days after the initial injection of the tuberculin, which is not always easy or convenient. Secondly, the results are subject to interpretation by the person undertaking the examination. Thirdly, interpretation is complicated in patients who have had BCG vaccine or who have been exposed to other mycobacteria as this may result in the presentation of false positives. Fourthly, the test is not particularly sensitive and so can also result in a number of false negatives (Chaturvedi N et al. 1992).

In the last several years, a new generation of cellular immune-based tests have been developed and are entering clinical practice. They measure ex vivo interferon-gamma T cell responses from blood samples after overnight incubation with protein antigens, notably ESAT-6 and CFP-10, that are present in M. tuberculosis but absent from BCG. Hence, test results are not confounded by prior BCG vaccination. Two commercially available assay formats exist: enzyme-linked immunospot (ELISpot) and enzyme-linked immunoassay (ELISA). The ELISpot assay was developed, patented and clinically validated by one of the inventors of the current invention. These assays appear to be a significant advance in the diagnosis of M. tuberculosis infection. Both assays are more specific than the skin test and the ELISpot is also more sensitive. However, while it is known that persons with positive skin test results after TB exposure have an increased risk of progression to active TB over the subsequent few years (thereby indicating that a positive skin test result reflects latent infection with dormant but viable bacilli that retain disease-causing capability), the prognostic value of a positive T cell-based interferon gamma (TIGRA) result in recent TB contacts is unknown. Hence, it is not yet certain whether positive TIGRA results indicate infection with dormant but still-viable bacilli with disease causing capability.

Both the skin test and the TIGRA tests suffer key disadvantages:

Firstly, they cannot differentiate active TB infection form latent TB infection.

Secondly, although TIGRAs are more dynamic than the skin test in that the strength of response declines with successful anti-TB therapy (Lalvani et al, J Inf Dis 2001; Pathan et al, J Immunol 2001; Lalvani et al am j resp crit care med 2001; Lalvani et al lancet 2001; Ewer et al Lancet 2003), there is very wide inter-individual variation in the decline and a substantial proportion of patients continue to have positive blood test results long after completion of treatment (Ewer et al, Am J Resp Crit Care Med 2006; Chee et al, Am J Resp Crit Care Med 2006; Millington et al, J Immunol 2007). Hence, early expectations that these assays, particularly ELISpot, could be used to monitor anti-TB treatment, or could be used as a test of cure, have not been fulfilled by longitudinal studies. This is likely because HLA class I and class II-restricted peptide-specific CD8 and CD4 memory T cells persist long after treatment, at a level sufficient to still give positive responses in TIGRAs (Millington et al J Immunol 2007).

Thirdly, they have limited diagnostic sensitivity, which limits the clinical usefulness of the tests to some extent. This is because the value of negative test results depends on very high diagnostic sensitivity in order to rule out a suspected diagnosis of TB.

It is an object of the present invention to provide an improved cellular immune-based test for identifying mycobacterial infection which overcomes the above limitations of the skin test and the TIGRAs.

According to the present invention there is provided a method of assessing mycobacterial infection in a subject comprising;

i. exposing at least one CD1 molecule or analogue to mycolic acid or a mycolic acid analogue;
ii. incubating the at least one CD1 molecule or analogue with a sample comprising at least one T cell isolated from the subject;
iii. measuring the T cell response and/or the number of mycolic acid specific T cells present in the T cell sample.

As used herein, the term assessing includes; diagnosing mycobacterial infection manifesting as TB; diagnosing latent mycobacterial infection which does not manifest as disease; differentiating between active and latent mycobacterial infection; and monitoring the progress or change in the status of mycobacterial infection over time. Wherein the change may occur spontaneously or as a result of treatment with a drug or vaccine or a test drug or test vaccine.

It will be further apparent to the skilled person that step ii. of the method may be performed concomitantly with or subsequently to step i.

In one embodiment, the at least one CD1 molecule or analogue comprises at least one dendritic. It will be understood by the skilled person that the use of dendritic cells to present the CD1 molecules is not essential to the present invention. CD1 molecules may be presented in any suitable manner known to those skilled in the art.

Dendritic cells are cells which form part of the immune system. These cells process antigenic material and present it on their surface for recognition by other cells of the immune system. They are found in an immature state in the blood and once activated migrate to the lymphoid tissue where they are involved in initiation and control of immune response.

In a preferred embodiment at least one dendritic cell is produced by culturing ex vivo at least one monocyte isolated from the subject.

Within the body, monocytes are produced from monoblasts in the bone marrow and released into the circulation where they circulate in the blood for 1 to 3 days before moving into the tissues of the body.

Alternatively, the at least one CD1 molecule or analogue may comprise an artificially synthesised CD1 molecule. This strategy designed to circumvent the requirement for autologous DC generation, would involve immobilisation of lipid-loaded CD1b monomers onto a substrate. In a preferred embodiment, the substrate is pvdf-coated substrate.

In a further preferred embodiment, lipid-loaded CD1b monomers can be immobilised on the surface of MHC class I and class II negative cells in the same manner used by Savage et al with MHC class I monomers (Ogg GS et al. 2000), incorporated herein by reference.

In a further alternative embodiment, the at least one CD1 molecule or analogue may be derived from a cell line expressing CD1 molecules. It will be apparent that this cell line could be used as a means of presentation.

Preferably, the CD1-expressing cell line is also MHC class I and class II negative. For example, De La Salle et al, incorporated herein by reference, have used MHC-positive THP-1 cells transfected with CD1b to activate CD1b-restricted T cell clones (de la Salle, Mariotti et al. 2005).

In a preferred embodiment the T cell response is compared to that seen upon contacting said T cell with dendritic cells not previously exposed to mycolic acid. It will be understood that an increase in the T cell response when contacted with dendritic cells exposed to mycolic acid compared to that seen in dendritic cells not exposed to mycolic acid indicates mycobacterial infection.

Preferably, the T cells are CD1 restricted T cells.

There are a number of advantages of using detection of mycolic acid T cell responses as a means to diagnose mycobacterial infection. Lipid antigens are presented by the CD1 molecules which are expressed in all humans and are not highly polymorphic like the MHCI and MHCII molecules which present peptide antigens (including ESAT-6 and CFP10). This means that antigen-presenting cells in all humans can potentially present mycolic acid, in contrast to protein antigens where presentation of peptide epitopes to T cells is limited by an individual's genetic make-up, i.e. tissue type or HLA haplotype. Therefore, a mycolic acid-based diagnostic test will allow high diagnostic sensitivity in out-bred genetically heterogeneous populations.

CD1 molecules themselves are a family of glycoproteins expressed on the surface of various human antigen presenting cells and are subdivided into group 1 and group 2 CD1 molecules. Group 1 CD1 molecules present foreign lipid antigens and specifically a number of mycobacterial cell wall components, to CD-1 specific T cells, making them particularly suitable for use in identifying mycobacterial infection (Manfred Brigl et al. 2004).

It is likely that the long-term immunological memory that is a hallmark of HLA class I and class II-restricted CD8 and CD4 peptide-specific T cells is quite different in CD1-restricted T cells, which represent a hybrid between innate and adaptive immunity. It follows that when CD1-restricted lipid antigens are cleared from the body, e.g. after successful anti-TB treatment, that CD1-restricted T cells will decline greatly in numbers, in contrast to peptide-specific memory T cells which persist at increased numbers for many years after treatment of infection (Millington et al, J Immunol 2007).

For these reasons, quantitative detection of lipid-specific CD1-restricted T cells (or their products) may differentiate between active TB disease (where bacterial burden is high) on the one hand and latent TB infection (where bacterial burden is low) on the other. Furthermore, it may also distinguish between untreated active TB and successfully treated TB infection (where it is believed that no bacteria remain). The methods of the current invention also allow monitoring of the levels of infection, whether active TB or latent infection, during treatment.

These would be significant advantages over TIGRAs and would imply that the current invention might replace protein-based TIGRAs or may work in a synergistic and complementary manner with the information they provide.

Because detecting T cells or T cell responses specific for mycolic acid may differentiate between active TB infection and latent infection and since no T cell-based diagnostic test developed to date has both high diagnostic sensitivity and reliably distinguishes active TB disease from latent TB infection the current invention provides a clear advantage over existing tests.

Mycolic acids themselves are long fatty acids found in the cell walls of the mycolata taxon of bacteria where they form the major component of the cell wall. Long mycolic acids possessing between 60-90 carbons are found in all the genera of Mycobacterium and therefore may be useful in diagnosing infections by other mycobacterium in sick individuals not presenting TB associated symptoms, including M. leprae and M. avium. M. tuberculosis produces three main types of mycolic acids Alpha-, methoxy- and keto, of which alpha-mycolic acids comprise at least 70% (Brenner et al. 1995).

In one embodiment, the T cells are in the form of peripheral blood lymphocytes (PBL's).

PBL's are mature lymphocytes that are found circulating in the blood, as opposed to being located in organs such as lymph nodes, spleen, thymus, liver or bone marrow.

In a further embodiment, T cells are isolated from body fluids taken from sites of active TB disease, for example bronchoalveolar lavage (lung washings) or pleural effusions or cerebrospinal fluid or ascites.

However, it will be understood that any body fluid containing T cells can be used in the methods of the current invention, which are not restricted to T cells from disease sites or blood. The envisaged body fluids include bronchial alveolar lavages (BAL), lung biopsy, sputum (including induced sputum), ascites, pleural fluid, pleural biopsy, lymph node biopsy, joint aspirate, cerebral spinal fluid, soft tissue abscess and any other affected part of the body.

Preferably, the T cell response measured is secretion of one or more cytokines and/or chemokines or expression of one or more markers of T cell activation.

Preferably, the cytokine is IFN γ. However, it will be apparent to the skilled person that other cytokines, for example TNF-α or IL-2, and/or chemokines, for example RANTES, MCP-1 or MIP1-α, can be employed in the method of the current invention.

It will be readily apparent to the skilled person that the cytokine or chemokine can be detected by any suitable technique known in the art, for example, ELISPOT or intracellular cytokine staining followed by flow cytometry, or cytokine secretion and capture assay or ELISA or whole-blood ELISA.

In an alternate embodiment, mycolic acid specific T cell numbers are measured. The skilled person will understand that this can be done by a number of methods well known in the art, for example tetramer or pentamer staining followed by flow cytometry (Klenerman P et al, Tracking T cells with tetramers: new tales from new tools, Nat Rev Immunol. [2002] 2(4):263-72).

It will further be apparent that the presence of mycolic acid specific T cells indicates mycobacterial infection.

Preferably, the mycobacterial infection is M. tuberculosis(TB) infection.

Preferably, the mycolic acid is isolated from mycobacteria. More preferably, mycobacterium is M. tuberculosis complex.

It will be obvious that the methods of the invention may be useful in any mammal.

In preferred embodiments, the methods are for use in medical and/or veterinary fields, for example in the diagnosis of mycobacterial infection in domesticated mammals including livestock (e.g. cattle, sheep, pigs, goats, horses or in wild mammals, such as those captive in zoos).

In the most preferred embodiment of the current invention, the subject is a human.

In a further preferred embodiment, the subject is receiving or has previously received a therapeutic intervention.

Preferably, the method further comprises comparing the status of infection to the previously determined status of said infection in said individual, thereby monitoring the effectiveness of said therapeutic intervention in said individual.

It will be readily apparent that the methods of the current invention can be used in combination with any previously known test for diagnosing Mycobacterial infection. For example, the current methods could be used to augment diagnostic sensitivity of existing T cell-based diagnostic tests of TB infection, e.g. those using protein antigens encoded in MTB Region of difference-1.

According to a further aspect of the present invention there is provided a product, combination or kit for assessing mycobacterial infection in a subject, comprising at least one CD1 molecule or analogue, and a T cell response detection means.

In a preferred embodiment said T cell response detection means is at least one antibody. More preferably, the antibody is specific for a cytokine, chemokine, or a marker of T cell activation or proliferation. Even more preferably, the antibody is a mAB.

It will be understood that any feature of the method of the first aspect can be incorporated into the product combination or kit of the second aspect.

The invention will now be further described with reference to the following example, in which:

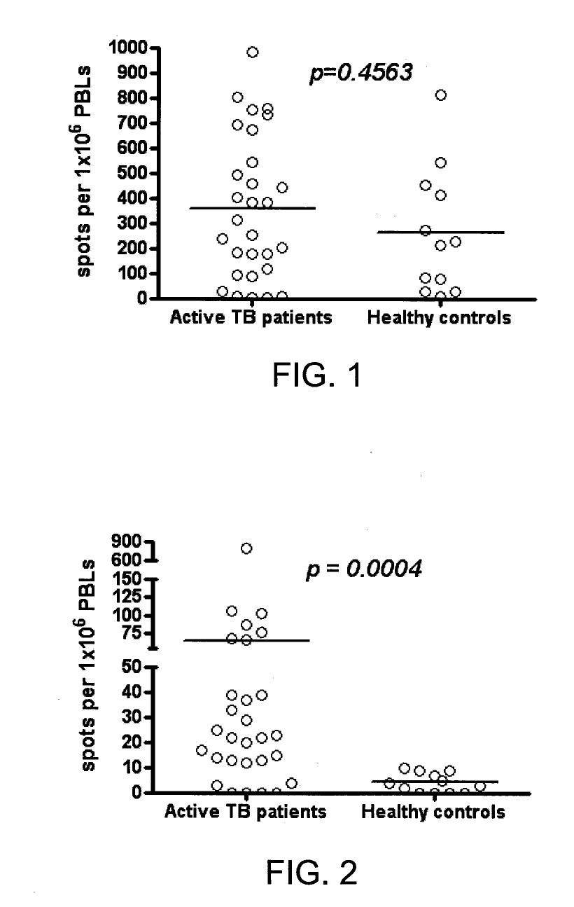

FIG. 1 shows the PPD response of healthy individuals compared to individuals infected with TB.

FIG. 2 shows the mycolic acid response of healthy individuals compared to individuals infected with TB.

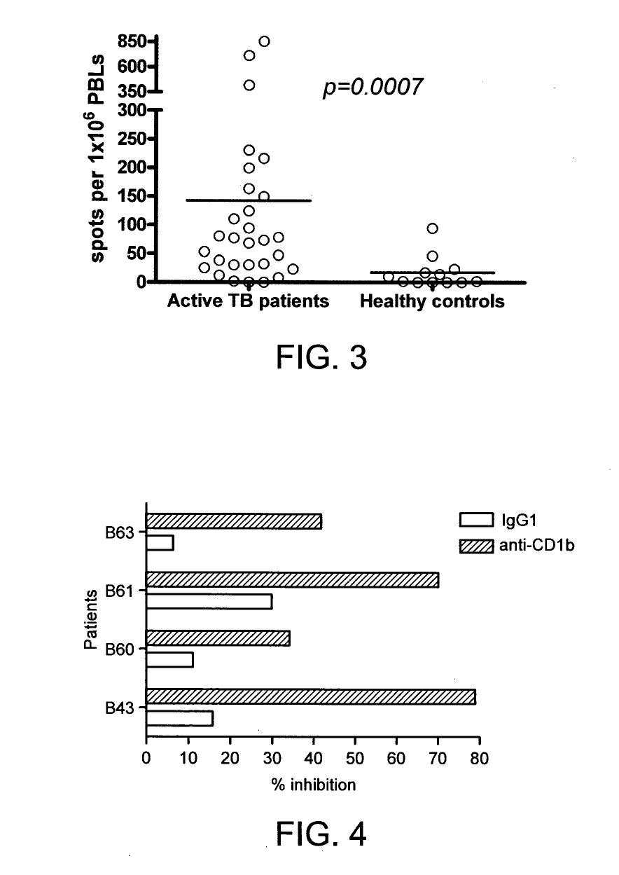

FIG. 3 shows the total M. tuberculosis lipid lysate response of healthy individuals compared to individuals infected with TB.

FIG. 4 shows blocking of mycolic acid-specific responses in TB patients by an anti-CD1b antibody.

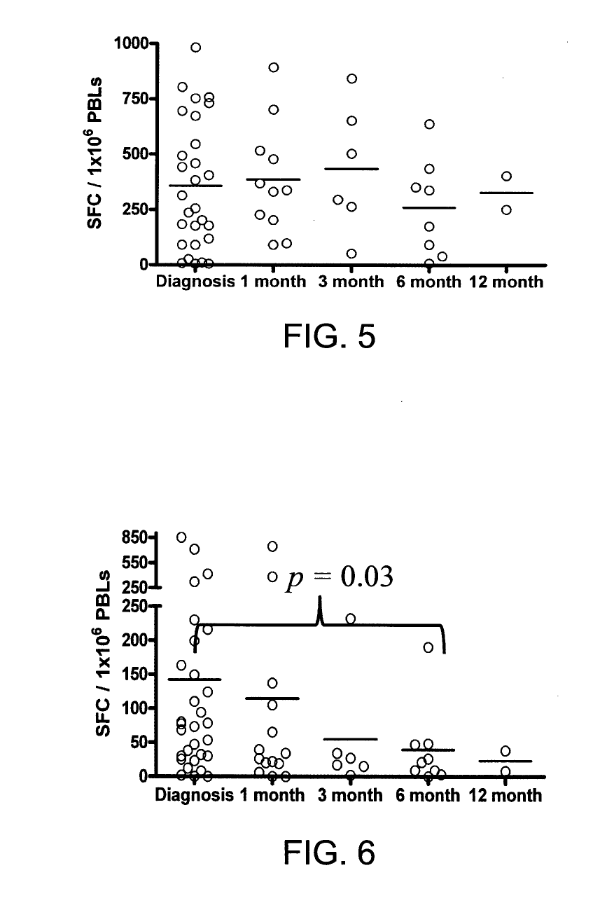

FIG. 5 shows the evolution of PPD-specific responses in TB patients during the period of treatment.

FIG. 6 shows the evolution of M. tuberculosis lipid lysate-specific responses in TB patients during the period of treatment

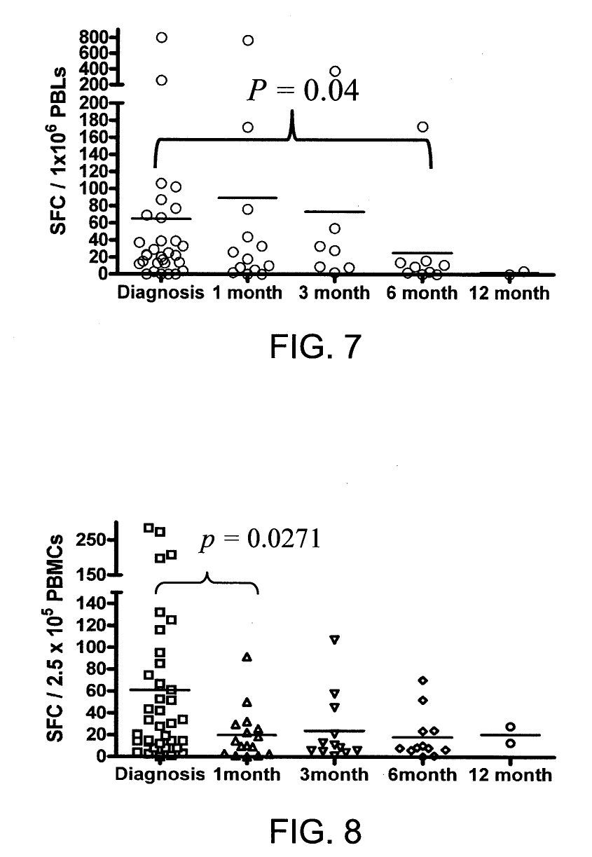

FIG. 7 shows the evolution of Mycolic acid-specific responses in TB patients during the period of treatment.

FIG. 8 shows the evolution of ESAT-6-specific responses in TB patients during the period of treatment.

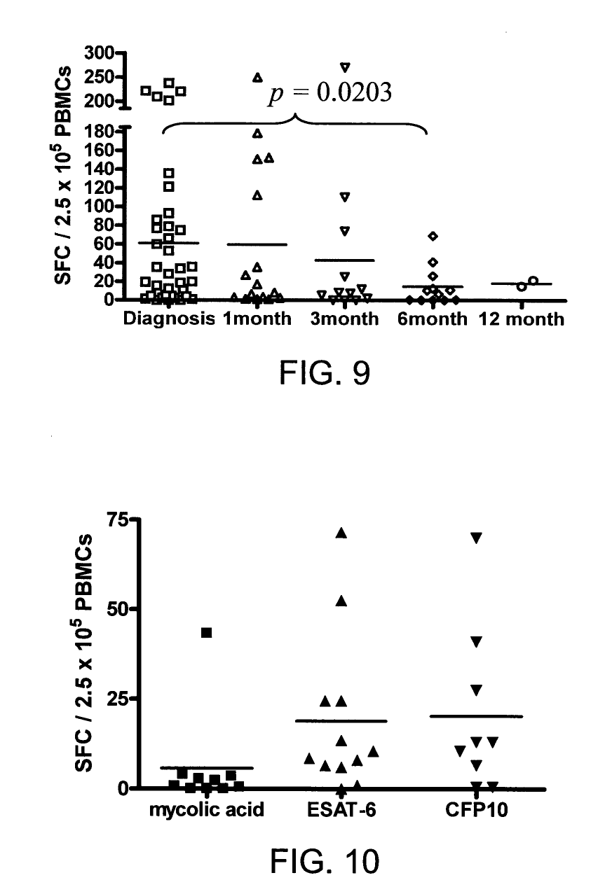

FIG. 9 shows the evolution of CFP10-specific responses in TB patients during the period of treatment.

FIG. 10 shows a comparison of mycolic acid-specific responses and ESAT-6 and CFP10-specific responses in TB patients after 6 month of treatment.

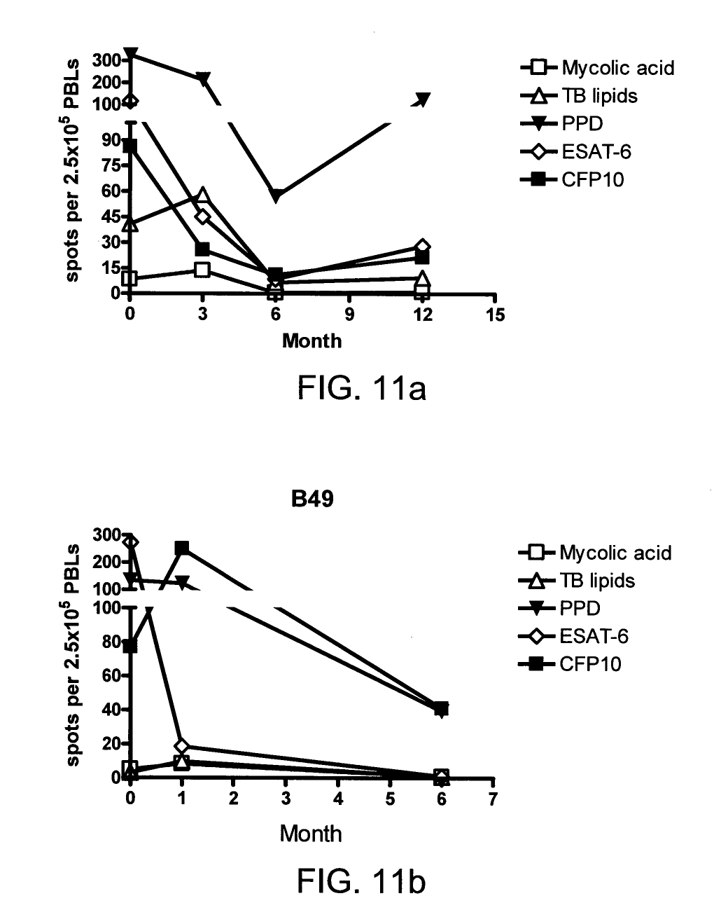

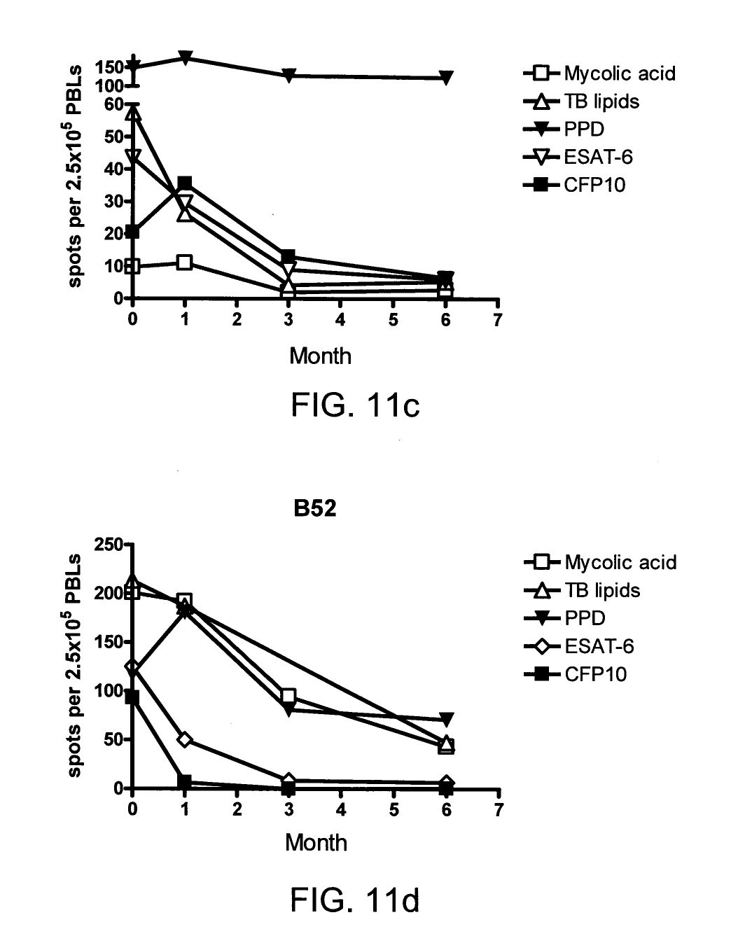

FIG. 11 shows the evolution of responses to PPD, mycolic acid, M. tb lipids, ESAT-6 and CFP10 in individual TB patients during the period of treatment.

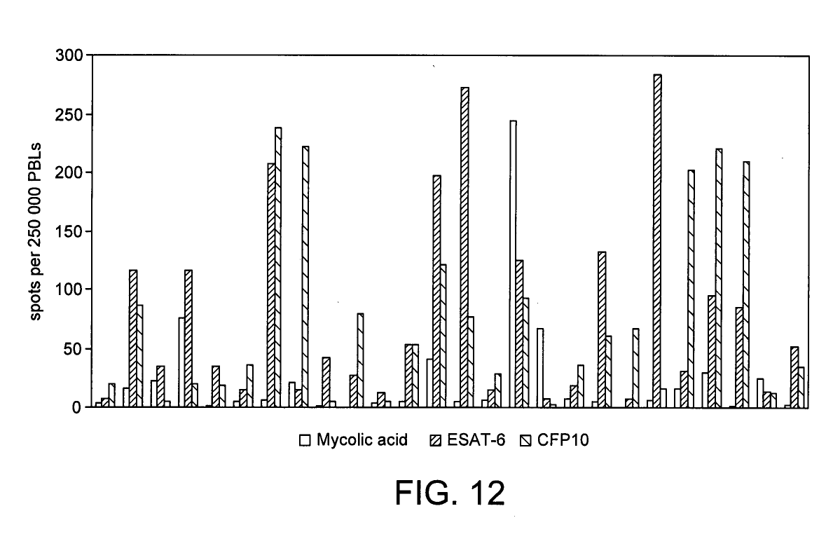

FIG. 12 shows responses to mycolic acid, ESAT-6 and CFP10 in TB patients at diagnosis.

### EXAMPLE 1

### Methodology

Operating protocol for the processing of blood samples from MTB infected patients and assessing T cell response

### Day 0

PBMCs (peripheral blood mononuclear cells) are isolated by density gradient centrifugation using Lymphoprep™ (AXIS-SHIELD UK ltd, Huntingdon, UK). Cells were then washed twice with RPMI 1640 medium and resuspended in a 2% Human serum media (RPMI 1640, 2% human serum, 2 mM L-glutamine and 100 U/ml penicillin-streptomycin) at 6×10⁶ cells/ml. The PBMCs are then incubated for one hour at 37° C. in medium culture flasks. Non-adherent cells are washed off with three washes of warm PBS (saline solution), and adherent cells are incubated at 37° C. overnight in 5% human serum media with GMCSF (at a 1:1000 dilution). The washed-off PBLs (peripheral blood lymphocytes) are frozen down in a 10:1 foetal-calf serum, DMSO solution at 15×10⁶ cells/ml at −80° C., and transferred into liquid nitrogen the next day.

### Day 1

Monocytes are harvested by washing with cold PBS and plated at 1×10⁶ cells per well on a 48-well culture plate at 2×10⁶/ml in 5% human serum media+IL-4 (1:1000)+GMCSF (1:1000). The ELISPOT plate is washed 6 times with PBS and 50 uL of a 1:200 7-B6-1 ALP detection antibody (Mabtech, Sweden) in PBS solution is added to the wells and left to incubate at room temperature for 90 min. After 6 washes with PBS 50 uL of the BCIP/NBT substrate is added to each well for 10 min. The plate is then washed under the tap and left to dry.

### Day 3

Monocytes are fed by adding 1 ml of 5% human serum+GMCSF (1:1000)

### Day 6

The cultured monocytes (now immature DCs) are pulsed with 4 ul of a 100 ug/ml solution of antigen solubilised in DMSO. The antigens used are PI, Mycolic acid, total TB lipid lysate and DMSO. Prior to pulsing of immature DCs, the lipids solubilised in DMSO are heated for 10 min in a 70° C. waterbath, to ensure full solubilisation.

### Day 7

The cultured DCs are harvested through multiple suction and resuspended in a 10% HS media at 1×106 cells/ml. PBLs from the same patient are thawed in a 37° C. waterbath and washed twice with RPMI, they are then resuspended in a 10% HS media at 10×106 cells/ml.

200 uL of 10% HS media is added to wells from a 96-well precoated PVDF (polyvinylidene fluoride) membrane plate coated with a cytokine specific capture mAb (monoclonal antibody) 1D1-k (Mabtech, Sweden). The plate is incubated at 37° C. for 60 min. The wells are emptied and 50 uL of the PBLs +100 uL of DCs are added to each well. The plate is left overnight at 37° C.

### Day 8

The ELISPOT plate is washed 6 times with PBS, and 50 uL of a 1:200 7-B6-1 ALP detection antibody (Mabtech, Sweden) in PBS solution is added to the wells and left to incubate at room temperature for 90 min. After 6 washes with PBS, 50 uL of the BCIP/NBT substrate is added to each well for 10 min. The plate is then washed under the tap and left to dry.

Operating protocol for the refolding of CD1 molecules Guanidine-denatured CD1b proteins were renatured by dilution refolding at 6-8° C. 500 ml of refolding buffer (100 mM Tris pH8, 400 mM L-arginine, 2 mM EDTA, 5 mM reduced glutathione, 0.5 mM oxidized glutathione and 0.1 mM PMSF) was prepared in a 500 ml beaker. The refolding buffer was stirred constantly using a magnetic stirrer. 5 mg of BPI was added to the refolding mix, by first pre-diluting the protein 1 in 5 with refolding buffer. After 45 min, 500 μM of the CTAB detergent was added to the refolding buffer followed by addition of 1.5 μg of lipid. Lipid was previously dried and resuspended in 1 ml of a vehicle solution (0.05% Tween, 20 mM NaCl), and heated for 10 min in a 60° C. water bath. 15 mg of denatured CD1b, diluted 1 in 5 with refolding buffer, was added in 5 aliquots over three days. 12 molar equivalents (relative to CTAB) of methyl-β-cyclodextrin (mCAB) were then added. Refolded protein was then concentrated with Amicon stirred cells to 7 ml, filtered through a 0.2 μM filter and biotinylated. The complex was then purified using gel filtration performed with a Pharmacia 26/60 Superdex 200 column in 20 mM Tris, 150 mM NaCl at pH 8, using the Pharmacia AKTA FPLC system.

The refolded CD1 molecules may then be used to assess T cell responses in the methods of the current invention.

### Results

In order to assess the accuracy of the method of the current invention, the response of T cells present in the PBL'S to mycolic acid was compared in a number healthy donors (BCG vaccinated) with no known exposure to M. tuberculosis and a number of active TB patients. Table 1 shows the demographic and clinical characteristics of the test subjects.

TABLE 1
Clinical and demographic data

| | | |
|---|---|---|
| | Active TB | Healthy |
| Characteristic | patients (%) | Controls (%) |

| Total | 30 | | 12 | |
|---|---|---|---|---|
| Age (years) | 38.25 | | 27.6 | |
| (median, range) | (22, 62) | | (21, 37) | |
| Male | 20 | (67%) | 7 | (64%) |
| Ethnicity | | | | |
| Indian sub- | 16 | (53%) | 3 | (18%) |
| Black African | 10 | (33%) | 0 | |
| Caucasian | 4 | (14%) | 9 | (82%) |
| BCG vaccinated | 16 | (53%) | 12 | (100%) |
| Site of Disease | | | | |
| Pulmonary | 17 | (57%) | | |
| Extra-pulmonary | 6 | (20%) | | |
| Lymph nodes | | | | |
| Musculoskeletal | 5 | (17%) | | |
| PSOAS Abcess | 1 | (3%) | | |
| Non-classified | 1 | (3%) | | |
| Positive M. tb | 22 | (73%) | | |

In order to compare the accuracy of the results for mycolic acid with those to other antigens, the responses to several of these were also measured. The other antigens measured were the lipid vehicle alone (DMSO) used as a negative control (data not shown), phosphotidylinositol (PI), also used as a negative control to verify that activation of T-cells was not only due to antigen processing and was lipid-specific, PPD and a total M. tuberculosis lipid lysate.

ESAT-6 and CFP10, two proteins from the RD-1 region of M. tb, a region deleted in BCG, and currently used in two different diagnosis tests (T-SPOT and Quantiferon-gold) were used as a basis for comparison. Phytohaemaglutinin (PHA) was used as a positive control in all experiments (data not shown).

T cell response was measured by enumerating IFN-γ Spot Forming Cells in both healthy controls and active TB patients.

The responses to mycolic acid, PPD and total M. tuberculosis lipid lysate were normalised to the PI response. The numbers represent the number of spots observed per 500 000 PBLs (peripheral blood lymphocytes). A cut-off point of 6 was selected by consideration of what value provides the best separation of data points for TB patients and healthy controls.

The data was subjected to a Mann Whitney analysis with the hypothesis that active TB patients would have a higher response than healthy controls.

FIG. 1 shows PPD responses observed in both healthy controls and active TB patients. When applying the Mann Whitney test, no statistically significant difference was observed between the two groups in response to challenge with PPD, p=0.4563.

However, FIG. 2 shows that mycolic acid-specific responses are statistically significantly higher in patients with active TB compared to healthy BCG-vaccinated controls (p=0.0004). Applying a cut-off value of 6 spots, over which responses are considered positive, yields high diagnostic sensitivity of 80% (24 out of 30 TB patients were positive) and high diagnostic specificity of 100% (12 out of 12 BCG-vaccinated healthy controls were negative).

T cell responses to total TB lipid lysate in both healthy controls and active TB patients were also measured and the results are shown in FIG. 3. When applying a Mann Whitney test with the hypothesis that active TB patients would have a higher response than the healthy controls, p=0.0007 is obtained.

A previous study (Ulrich et al 2003) showed a statistically significant difference in T cell responses between persons with presumed latent TB infection (PPD+) and healthy controls presumed to be uninfected (PPD−) when using a total M. tuberculosis lipid lysate as the target antigen. Our results suggest that a similar difference in the IFN-γ T cell response to whole lipid lysate exists between healthy uninfected individuals and active TB patients. However, quantitative responses to a total M. tuberculosis lipid lysate is less specific, with 5 uninfected controls scoring positive.

Thus mycolic acid is a more advantageous antigen to use in a TB diagnostic test.

Using an anti-CD1b antibody, it was ascertained that the mycolic acid-specific response is CD1b restricted, see FIG. 4.

Responses to M. tb lipids, mycolic acid, PPD, ESAT-6 and CFP10 were followed during treatment. FIGS. 6 and 7 show that responses to M. tb lipids and mycolic acid, respectively, are reduced during the period of treatment, giving a statistical difference of p=0.03 and p=0.04 respectively between diagnosis and 6 months of treatment, where as PPD-specific responses remain stable, as shown in FIG. 5. In the case of mycolic acid, responses reach undetectable levels in most patients at the 6 month time point and appear to remain so 12 months after diagnosis.

This reduction in the magnitude of responses during treatment can also be observed with ESAT-6 and CFP10, see FIGS. 8 and 9 respectively. However, most patients tend to still have a positive response to either of these two protein antigens after 6 months of treatment and these responses appear to remain detectable up to 12 months after diagnosis. When directly comparing mycolic acid, ESAT-6 and CFP10 responses at 6 months after diagnosis, most patients do not respond to mycolic acid, while ESAT-6 and CFP10 responses tend to remain detectable, see FIG. 10. Therefore, mycolic acid-specific responses may be useful in the diagnosis of active disease.

When following individual TB patients over the period of treatment, see FIG. 11, it is seen that 3 out of 4 patients (B16, B49 and B55) no longer respond to mycolic acid after 6 months of treatment while still having detectable responses to either ESAT-6 or CFP10. In the case of patient B16, the mycolic acid specific response remains undetectable up to 12 months after diagnosis where as ESAT-6 and CFP10-specific responses remain detectable. In patient B52, the mycolic acid-specific response is still visible after 6 months of treatment, however the magnitude of the mycolic acid-specific response at diagnosis was unusually high when compared to other TB patients. Hence it is suggested that this response may take longer to disappear, reaching undetectable levels by the 12 month time point. These results provide a further indication that the presence of mycolic acid-specific responses may be a useful marker of active disease.

When directly comparing mycolic acid-specific responses to ESAT-6 and CFP10 responses at diagnosis, it is observed that a strong response to one antigen does not correlate with a strong response to either of the other two, see FIG. 12. This indicates that combining all three antigens might improve on the sensitivity of current diagnostic tests.

In the cohort of patients used in the current study it was found that ESAT-6 and CFP10-specific responses are more predominant then mycolic acid-specific responses, with 4 patients responding to ESAT-6 but not mycolic acid and 3 patients responding to CFP10 but not mycolic acid. One patient was also found to respond to mycolic acid while having a negative CFP10-specific response, see Table 2.

TABLE 2

| | ESAT-6 positive | CFP10 positive |
|---|---|---|
| Mycolic acid | 22 | 21 |
| positive | | |
| Mycolic acid | 4 | 3 |
| negative | | |

For the avoidance of doubt, it will be understood that all references cited are incorporated herein in their entirety.

### REFERENCES

Brenner P. J, Nikaido H. The envelope of mycobacteria. Annu Rev biochem. [1995] Volume 64, page 29-63.
Chaturvedi N, Cockcroft A. “Tuberculosis screening among health service employees: who needs chest X-rays?”. J Soc Occup Med [1992] 42: 179-82.
Chee et al, Am J Resp Crit Care Med. [2007], Feb. 1; 175(3):282-7.
De la Salle et al, Science. (2005) Nov. 25; 310(5752):1321-4
Dinnes et al, Health Technol Assess. [2007] January; 11(3):1-196.
Ewer et al, Lancet [2003] Apr. 5; 361(9364):1168-73.
Ewer et al, Am J Resp Crit Care Med. [2006] Oct. 1; 174(7):831-9.
Lalvani et al, J Inf Dis. [2001] Feb. 1; 183(3):469-77.
Lalvani et al, am j resp crit care med. [2001] March; 163(4):824-8.
Lalvani et al, lancet [2001] Jun. 23; 357(9273):2017-21.
Manfred Brigl, Michael B. Brenner. CD1: Antigen Presentation and T Cell Function. Annual Review of Immunology [2004] Volume 22, Page 817-890, Apr.
Millington et al, J Immunol [2007] Apr. 15; 178(8):5217-26.
Ogg GS et al, Br J Cancer (2000) March; 82(5):1058-62.
Pathan et al, J Immunol [2001] Nov. 1; 167(9):5217-25.
Ulrichs, T. Moody, D. B. Grant, E. Kaufmann, S. H. Porcelli, S. A. T-cell responses to CD1-presented lipid antigens in humans with Mycobacterium tuberculosis infection, Infect Immun. [2003] Volume 71, Issue 6, page 3076-87, Jun.
World Health Organization. WHO Report 2006. Global tuberculosis control.

1. A method of assessing mycobacterial infection in a subject comprising;
i. exposing at least one CD1 molecule or analogue to mycolic acid or a mycolic acid analogue;
ii. incubating the at least one CD1 molecule or analogue with a sample comprising at least one T cell isolated from the subject; and,
iii. measuring the T cell response and/or the number of mycolic acid specific T cells present in the T cell sample.

2. The method according to claim 1, wherein the at least one CD1 molecule or analogue comprises at least one dendritic cell.

3. The method according to claim 2, wherein the at least one dendritic cell is produced by culturing ex vivo at least one monocyte isolated from the subject.

4. The method according to claim 1, wherein the at least one CD1 molecule or analogue comprises an artificially synthesised CD1 molecule.

5. The method according to claim 4 wherein the CD1 molecule is immobilised on a substrate.

6. The method according to claim 1, wherein the at least one CD1 molecule or analogue is derived from a cell line expressing CD1.

7. The method according to claim 1, wherein the T cells are CD1 restricted T cells.

8. The method according to claim 1, wherein the T cells are peripheral blood lymphocytes (PBL's).

9. The method according to claim 1, wherein the T cells are isolated from a disease site or suspected disease site.

10. The method according to claim 1, wherein the T cell response is compared to that seen upon contacting of said T cells with dendritic cells not previously exposed to mycolic acid or a mycolic acid analogue.

11. The method according to claim 1, wherein the T cell response measured is secretion of one or more cytokines and/or chemokines, or expression of one or more markers of T cell activation or proliferation.

12. The method according to claim 11, wherein the T cell response is secretion of IFN γ.

13. The method according to claim 11, wherein an increase in response indicates mycobacterial infection.

14. The method according to claim 1, wherein the number of mycolic acid specific T cells in the T cell sample are counted by CD1 tetramer/pentamer staining.

15. The method of claim 14, wherein the presence of mycolic acid restricted T cells indicates mycobacterial infection.

16. The method according to claim 1, wherein the mycolic acid is isolated from mycobacteria.

17. The method according to claim 1, wherein the mycobacterial infection is M. tuberculosis(TB) infection.

18. The method according to claim 17, wherein said method can distinguish between active and latent TB infection.

19. The method according to claim 16, wherein the mycobacterium is M. tuberculosis complex.

20. The method according to claim 1, wherein the subject is a mammal.

21. The method according to claim 1, where the subject is receiving or has previously received a therapeutic intervention.

22. The method according to claim 21, further comprising comparing the status of infection to the previously determined status of said infection in said subject, thereby monitoring the effectiveness of said therapeutic intervention in said subject.

23. A product, combination or kit for assessing mycobacterial infection in a subject comprising at least one CD1 molecule or analogue, and a T cell response detection means.

24. The product, combination or kit according to claim 23, wherein said T cell response detection means is at least one antibody.

25. The product, combination or kit of claim 24, wherein the at least one antibody is specific for a cytokine, chemokine, or markers of T cell activation or proliferation.
